(19) United States
(12) Patent Application Publication
(10) Pub. No.: US 2003/0113805 A1
(43) Pub. Date: Jun. 19, 2003

(54) Intrinsically labelled solid support

(75) Inventors: Brian Geoffrey Main, Macclesfield (GB); Richard Eden Shute, Macclesfield (GB)

Correspondence Address: Janis K. Fraser, Ph.D. J.D, Fish & Richardson P.C., 225 Franklin Street, Boston, MA, 02110-2804, US

(73) Assignee: Zeneca Limited, a London corporation

(21) Appl. No.: 10/339,552

(22) Filed: Jan. 9, 2003

Related U.S. Application Data
(63) Continuation of application No. 09/000,125, filed on Jan. 21, 1998, now abandoned.
Application No. 09/000,125 is a 371 of international application No. PCT/GB96/01696, filed on Jul. 17, 1996.

(30) Foreign Application Priority Data
Jul. 22, 1995 (GB) 9515070.2

(51) Int. Cl. G01N 33/53; G01N 33/543

(52) U.S. Cl. 435/7.1; 436/518

(57) ABSTRACT

A compound library comprises a plurality of different units each comprising a solid support with which is associated a single member of the compound library, each solid support has a defined chemical composition which acts as an intrinsic label capable of identifying the first choice in the synthesis of the associated member of the compound library.

The present invention relates to compound libraries synthesised on solid supports provided with intrinsic labels, a method for the production of said compound libraries, a method for the characterisation of members of said compound libraries which involves identifying the compounds by reference to the intrinsic labels of the associated solid supports and kits of intrinsically labelled solid supports for use in compound library synthesis.

The increasing demand for compounds which modulate biological processes, for example in medicine and agriculture, has lead to the development of methods for simultaneously synthesising a multiplicity of different compounds which can then be tested for characteristics of interest. Such compound libraries may be assembled in a number of ways, including the combine/mix/divide process described by Furka et al (Abstr. 14th Int. Congr. Biochem., Prague, Czechoslovakia, 1988, 5, 47; Int. J. Pept. Prot. Res., 1991, 37, 487-493), in this process libraries are created on polymer beads, such that each bead contains one discrete chemical species. The individual components of the library may be tested either still attached to the polymer bead on which they were synthesised (Lam et al, Nature, 1991, 354, 82-84) or after cleavage from the bead (Salmon et al, Proc. Nat. Acad. Sci. USA, 1993, 11708-11712). If tested while attached to the bead, or cleaved but physically associated with the bead, it is necessary to devise a method of identifying the library compound which is bound to a bead found to be active in the test. Where this compound is a polypeptide identification may be achieved by Edman degradation, either directly or after cleavage from the bead (Lam et al, Bioorg. Med. Chem. Lett., 1993, 419424); oligonucleotides may be identified by microsequencing techniques (Dower et al, Ann. Rep. Med. Chem., 1991, 26, 271-280).

Researchers have attempted to identify peptides containing unnatural amino acids (which are not amenable to Edman degradation) by co-synthesising a second peptide chain comprising natural amino acids and using this as a sequenceable code (Nikolaiev et al, Peptide Research, 1993, 6, 161-170); others have used oligonucleotide chains as cedes to identify the library compounds (Needels et al, Proc. Nat. Acad. Sci. USA, 1993, 90, 10700-10704 and WO 93/20242). The physical separation of the library compound and the coding peptide, whereby the library compound is displayed on the surface of the solid support and the coding peptide is synthesised on the interior of the bead, has also been suggested (Vágner et al in Innovation and Perspectives in Solid Phase Synthesis, R. Epton, Ed., Mayflower Worldwide Limited, Birmingham, 1994, 347-352). Identifier molecules such as mixtures of halogenated acids have also been used, these are bound to the bead in trace amounts at each stage of the synthesis thus forming an identifiable binary or higher order tagging system which defines the reaction history of the bead (Borchardt and Still, J. Am. Chem. Soc., 1994, 116, 373-374 and WO 94/08051). These methods utilising pendant labels have been reviewed extensively (Jacobs and Fodor, TIBTECH, 1994, 12, 19-26; Pavia et al Eds., Bioorg. and Med Chem. Lett., 1994, 381-470; Moos et al, Ann. Rep. Med. Chem., 1993, 28, 315-324; and Gordon et al, J. Med Chem., 1994, 37, 1233-1251 and 1386-1401).

Solid supports which are distinguishable by their colour, fluorescence, specific gravity or size have also been suggested (WO 93/06121 and WO 93/24517), for example pre-coloured supports have been prepared by coupling amino acid type dyes to aminomethylated polystyrene resin (Câmpian et al in Innovation and Perspectives in Solid Phase Synthesis, R. Epton, Ed., Mayflower Worldwide Limited, Birmingham, 1994, 469-472).

We have now devised a novel method for labelling solid supports wherein, rather then coupling one or more pendant labels to a solid support, an intrinsic label is incorporated into the support prior to its use in compound library synthesis. Thus by varying, in a controlled manner, the chemical composition of the support during manufacture we have been able to provide a large number of physically similar but chemically different solid. supports, the chemical composition of each solid support defining an intrinsic label.

The provision of solid supports having intrinsic labels has several advantages over prior art labelling methods including the possibility of manufacturing large quantities of intrinsically labelled supports prior to library synthesis thus increasing efficiency as no label has to be introduced concomitantly with the first reaction choice in the synthesis. In addition intrinsic labels may be designed to be inert to a greater variety of synthetic conditions than pendant labels thus extending the range of chemistry which can be utilised in library synthesis. Also, when library compounds are tested for characteristics of interest whilst still attached to the solid support, intrinsic labels avoid the possibility of a pendant label adversely affecting the test or of some co-operative interaction between the pendant label and the library compound resulting in false positive result

Thus, according to a first aspect of the invention, we provide a compound library comprising a plurality of different units each comprising a solid support with which is associated a single member of the compound library, characterised in that each solid support has a defined chemical composition which acts as an intrinsic label capable of identifying the first reaction choice in the synthesis of the associated member of the compound library.

The compound libraries according to the invention may comprise any convenient number of individual members, for example tens to hundreds to thousands to millions etc, of compounds. Suitable compounds include, for example, peptides, peptoids and other oligomeric compounds (cyclic or linear), and template-based smaller molecules, for example benzodiazepines, hydantoins, biaryls, polycyclic compounds (e.g. naphthalenes, phenothiazines, acridines, steroids etc), carbohydrate and amino acid derivatives, dihydropyridines, benzhydryls and heterocycles (e.g. triazines, indoles etc).

The compound libraries preferably comprise chemical compounds of low molecular weight which have potential as therapeutic agents. Such compounds are for example of less than about 1000 daltons, such as less than 800, 600 or 400 daltons.

Any convenient solid support may be used provided that it is inert, that is to say its mechanical integrity and chemical composition is not affected by the rigours of library synthesis and testing to any relevant extent. The solid support may have any convenient structure and shape. Suitable solid supports include beads, pellets, discs, capillaries, hollow fibres, needles, solid fibres etc. Beads represent a particularly convenient solid support for use in compound library synthesis, such as combine/mix/divide processes. The beads may be made of any material having a rigid or semi-rigid surface such as cellulose, pore-glass, silica gel, polystyrene resin, polystyrene cross-linked with divinyl benzene, grafted co-polymers such as polyethylene glycol/polystyrene, polyacrylamide, latex, dimethylacrylamide particularly cross-linked with N,N′-bis-acryloyl ethylene diamine and comprising N-t-butoxycarbonyl-β-alanyl-N′-acryloyl hexamethylene diamine. The beads may also be composites such as glass particles coated with a hydrophobic polymer such as cross-linked polystyrene or a fluorinated ethylene polymer to which is grafted linear polystyrene. The above resins are given by way of illustration only and further suitable solid supports will be apparent to the artisan of ordinary skill.

The solid support preferably comprises a polystyrene resin such as a polystyrene resin containing convenient reacting groups, e.g. amino, hydroxy or carboxy groups. Chloromethylpolystyrene resins such as chloromethylpolystyrene beads are a preferred solid support.

The incorporation of the intrinsic label into the solid support is achieved by varying, in a controlled manner, the chemical composition of the support during manufacture. The chemical composition of the support is preferably varied by the incorporation of quantities, for example up to 20% w/w, preferably 5-10% w/w, of at least one identifiable chemical compound into the material of the solid support. The identifiable chemical compounds are preferably distributed uniformly throughout the support Suitable identifiable chemical compounds include substituted styrenes. Preferred styrenes include those with inert substituents such as halostyrenes, e.g. fluoro-, chloro, bromo- or iodostyrene, and protected hydroxystyrenes, e.g. trifluoromethanesulphonyloxystyrene. Particularly preferred styrenes are the halostyrenes. Chloromethylpolystyrene beads, for example, containing intrinsic labels may be prepared by incorporating small amounts, for example up to 20% w/w, preferably 5-10% w/w, of simple styrenes containing inert substituents into the mixture of chloromethylstyrene, styrene and divinylbenzene commonly used for polymerisation to form beads for library synthesis. If an equimolar mixture of two differently substituted styrenes is used, each for example in a quantity of up to 10% w/w, then a large number of combinations and hence differently labelled solid supports can be obtained using a relatively small number of substituted styrenes.

During compound library synthesis a library is created, for example by the combine/mix/divide or split synthesis technique. Subsequent isolation of a solid support which has an associated library compound which exhibits a characteristic of interest, e.g. biological activity, followed by identification of the intrinsic label from the support allows identification of the portion of support used for the first stage of the synthesis and hence the nature of the first reaction choice, e.g. the first substituent introduced, in the synthesis of the associated member of the compound library. Identification of the last reaction choice, e.g. the last substituent introduced, by reference to the reaction which produced it, will also be possible if no ‘mix’ stage is performed. Knowing these two (first and last) reaction choices limits the number of possible structures for the member of the compound library to a level where it may be identified by, for example, mass spectrometry or iterative resynthesis. Synthesis of the compound libraries on the intrinsically labelled solid supports may comprise any convenient number of individual reaction steps.

Thus, according to a further aspect of the invention, we provide a method for the synthesis of a compound library comprising a plurality of different units each comprising a solid support with which is associated a single member of the compound library, which method comprises:

a) apportioning solid supports among a plurality of reaction vessels such that each reaction vessel contains a portion of solid supports having a unique defined chemical composition which acts as an intrinsic label capable of identifying the first reaction choice in the synthesis of the associated member of the compound library;

b) exposing the supports in each reaction vessel to a first reaction choice;

c) pooling the supports;

d) apportioning the supports among a plurality of reaction vessels;

e) exposing the supports in each reaction vessel to a further reaction choice; and

f) repeating steps c), d) and e) as required.

It will be appreciated that in the synthesis of a compound library according to the invention the chemical composition of one portion of the solid support may be defined by the fact that it contains no additional labelling compounds other than that of the support material itself.

In addition to the intrinsic labels as hereinbefore defined the units comprising the compound libraries of the invention may also contain one or more inert secondary labels which are introduced during library synthesis by controlled chemical modification of the solid support. These secondary inert labels are capable of identifying an intermediate reaction choice in the synthesis of an associated member of the compound library.

Convenient groups for controlled chemical modification of the solid support may be determined without undue experimentation. Particular groups are bromo and iodo, these may be present as substituents on substituted monomers, such as substituted styrenes, contained in the solid support. A preferred substituent is halo, e.g. bromo. It will be appreciated that convenient groups for modification may correspond to groups which form part of the intrinsic label, or other groups present in the solid support. We refer to such groups hereinafter as latent groups, that is to say they are inert with respect to compound library synthesis but may participate in the introduction of one or more inert secondary labels during library synthesis.

Particularly preferred intrinsic labels comprising latent groups are bromostyrene and iodostyrene. Preferred inert secondary inert labels include substituted monomers such as substituted styrenes produced by modification of latent groups on the intrinsic label. Suitable styrene substituents include vinyl, aryl e.g. phenyl, heterocyclyl and bicyclyl, and substituted aryl e.g. substituted phenyl, heterocyclyl and bicyclyl. Suitable aryl substituents include chloro, fluoro, alkyl (C1-C4), alkoxy, trifluoromethyl and mixtures of these, resulting in for example a (methylfluorophenyl)styrene label. Particular secondary labels which may be mentioned include 4(4′-chlorophenyl)styrene, 4-4′-trifluoromethyl-phenyl)styrene, 4-(4′-methoxyphenyl)styrene, 4-(1′-naphthyl)styrene.

The particular chemistry which is used selectively to modify these latent groups and so introduce the secondary labels constitutes a further aspect of the invention. The inert secondary labels are conveniently introduced during library synthesis by, for example, such procedures as protecting group removal followed by acylation or alkylation. In the case of bromostyrene and/or iodostyrene, particularly bromostyrene resins by a metal-catalysed cross-coupling reaction, e.g. a palladium-, nickel- or copper-catalysed cross-coupling reaction with an organometallic reagent, for example an arylboronate, organostannane, organozinc or organolithium reagent, in a so-called Heck, Stille or Suzuki coupling reaction (see for example Deshpande, Tetrahedron Lett., 1994, 35, 5613-4; Yu et al, Tetrahedron Lett., 1994, 35, 8919-22; Frenette & Friesen, Tetrahedron Lett., 1994, 35, 9177-80; and Forman & Sucholeiki, J. Org. Chem., 1995, 60, 523-8). If an equimolar mixture of two secondary labels is used then a large number of combinations is possible for a relatively small number of secondary labels.

The preferred synthesis of the secondary label is via a metal-catalysed cross-coupling reaction. Particularly preferred is the palladium-catalysed cross-coupling of an arylboronic acid to a latent group, in a so-called Suzuki coupling, where the intrinsic label is one of bromostyrene, iodostyrene or trifluoromethanesulphonyloxystyrene, the latter derived from protected hydroxystyrene.

Chloromethylpolystyrene beads, for example, containing intrinsic labels may be prepared by incorporating small amounts, for example up to 20% w/w, preferably 5-10%, of simple styrenes containing inert and latent substituents into the mixture of chloromethylstyrene, styrene and divinylbenzene commonly used for polymerisation to form the resin beads for library synthesis. If an equimolar mixture of two styrenes containing inert substituents and one styrene containing a latent group is used then a large number of combinations and hence differently labelled solid supports can be obtained using a relatively small number of substituted styrene labels.

During compound library synthesis a library is related, for example by the combine/mix/divide or split synthesis technique. Subsequent isolation of a solid support which has an associated compound which exhibits a characteristic of interest, e.g. biological activity, followed by identification of the intrinsic label from the support allows identification of the batch of support used for the first stage of the synthesis, and hence the nature of the first reaction choice, e.g. the first substituent introduced, in the synthesis of the associated member of the compound library. Identification of the secondary inert label(s) from the support allows the identification of the reaction choice from an intermediate round of the synthesis of the associated member of the compound library. Identification of the last reaction choice, e.g. the last substituent introduced, by reference to the reaction which produced it, will also be possible if no ‘mix’ stage is performed. Knowing these three (first, intermediate and last) reaction choices limits the number of possible structures for the member of the compound library to a level where it may either be identified directly, or, in the case of there being more than two ‘mix’ steps in the combine/mix/divide or split synthesis, by, for example, mass spectrometry or iterative resynthesis.

According to a further aspect of the invention we provide a method for the characterisation of members of a compound library comprising a plurality of different units each comprising a solid support with which is associated a single member of the compound library, which method comprises:

i) synthesising the library by a method comprising:

a) apportioning solid supports among a plurality of reaction vessels such that each reaction vessel contains a portion of solid support having a unique defined chemical composition which acts as an intrinsic label capable of identifying the first reaction choice in the synthesis of the associated member of the compound library;

b) exposing the supports in each reaction vessel to a first reaction choice;

c) pooling the supports;

d) apportioning the supports among a plurality of reaction vessels;

e) exposing the supports in each reaction vessel to a further reaction choice; and

f) repeating steps c), d) and e) as required;

ii) testing members of the compound library for a characteristic of interest;

iii) selecting library compounds of interest; and

iv) identifying the intrinsic labels of the associated solid supports and identifying the first reaction choice in the synthesis of such compounds by reference to said intrinsic labels.

The test for the characteristic of interest may be a biological assay which tests the ability of library compounds to modulate, in a test system, the activity of a biological of interest. Convenient biologicals of interest include proteins such as enzymes, receptors, and the like. Suitable test systems will be apparent to the scientist of ordinary skill. Any convenient number of library compounds may be tested in the biological assay.

The testing of the members of the compound library may be conducted whilst the compounds are still attached to the solid supports, alternatively the library compounds may be cleaved from the solid supports prior to testing.

The intrinsic and optional inert secondary labels are conveniently excised from the resin by depolymerisation, for example thermal depolymerisation, prior to identification. They can then be identified by a technique such as mass spectrometry (ms) or gas chromatography (gc). A combined process such as gc/ms may also be used.

By way of example, a single labelled polystyrene support is placed into the probe of a convenient mass spectrometer (for example magnetic sector, quadrupole, ion trap, or time of flight) and heated to a temperature of 500° C. During the course of the heating spectra are obtained continuously and the decomposition of the polymer monitored by the total ion chromatograph and/or alternately the characteristic styrene monomer and dimer ions that constitute the bulk of the spectrum (m/z 77, 78, 91, 103, 104, 105, 115, 117, 118, 165, 178, 193, 207). The spectra are summed over the peak of the evolution profile to give one spectrum which is displayed over a limited mass range from 119 to 400. The label may then be identified by ions at the m/z corresponding to the mono-isotopic molecular weight of the compounds, e.g. substituted styrene monomers, that have been incorporated into the support. Furthermore it should be noted that if the label comprises compounds which do not contain nitrogen then the ion at the m/z corresponding to the mono-isotopic molecular weight of the substituted styrene monomer will always possess an even mass number and its associated benzyl/tropylium ion 13 mass units (daltons) lower will thus always possess an odd mass number. This “fingerprint” distribution of molecular ions in the summed mass spectrum is further evidence of the label employed.

Alternatively a resin support is depolymerised by rapid heating using Curie point pyrolysis either on a mass spectrometer specifically designed for this purpose (for example Horizon instruments are suitable) or at the inlet to a gas chromatograph directly linked to a mass spectrometer. In the latter instance labels are identified by a combination of their mass spectra and their gas chromatographic retention times. Depolymerisation may also be achieved off-line from either mass spectrometer or gas chromatograph linked to a mass spectrometer by a combination of slow or rapid heating.

If desired the solid support may be divided into sections prior to chemical analysis, this can be accomplished by techniques such as milling, or preferably by microtomy. The latter technique can be performed at room temperature or at temperatures as low as −20° C. to −70° C. using a freezing microtome such as a Cryostat freezing microtome. In this technique a solid support associated with a library compound of interest is selected and mounted on or in a suitable support, for example a hard wax such as paraffin wax or beeswax; a resin such as polymethacrylate; a gum such as acacia; a suitable medium which can be frozen for use in a freezing microtome e.g. water, gelatin or concentrated sugar solutions; or any other support medium apparent to the scientist of ordinary skill, for example any organic solvent whose melting point is in the range −5° C. to +25° C. Examples of suitable organic solvents for freezing microtomy are tertiary butanol, 1,4-dioxane and acetic acid. Once mounted in the support medium, a handheld microtome, or other suitable microtome instrument, for example a Cryostat can be used to pare, portion or slice the solid support into sections of suitable thickness. Sections of thickness less than about 200 microns, such as less than 100, 50, 10 or 5 microns are preferred. Once portioned, the sections can be recovered from their support medium, in the case of freezing microtomy by thawing the support and recovering the portions, then analysed, treated or archived as required.

A kit of intrinsically labelled solid supports, such as labelled beads, as hereinbefore defined is novel.

Thus according to a further aspect of the invention, we provide a kit of solid supports for compound library synthesis compound library comprising a plurality of portions of a solid support upon which associated members of a compound library can be synthesised, characterised in that each portion of the solid support has a unique defined chemical composition which acts as an intrinsic label capable of identifying the first reaction choice in the synthesis of the associated member of the compound library.

In general such labelled supports are provided in sufficient quantity for use in the synthesis of compound libraries. Any convenient weight of material may be provided, such as for example up to 10, 100 or up to 1000 g of material. The maximum weight of material is limited only by practical considerations.

The invention will now be illustrated but not limited by the following Examples in which unless otherwise stated:

i) yields are given for illustration only and are not necessarily the maximum attainable by diligent process development;

ii) the following abbreviations have been used for particular organic solvents: THF for tetrahydrofuran and DME for dimethoxyethane.

### EXAMPLE 1

Preparation of Labelled Polystyrene Beads by Suspension Polymerisation Polymerisation

Polyacrylic acid Mn ˜70,000 (5 g), sodium sulphate (0.4 g) and distilled water (600 g) were added to a 1 litre 4 necked flask fitted with a stirrer, condenser and nitrogen inlet. The temperature of the flask was raised to 50° C. using a water bath with minimum stirring, maintaining a nitrogen blanket throughout. The stirrer speed was raised to 450 rpm and the following monomer mixture added in a thin stream:

| | |
|---|---|
| 4-chloromethylstyrene | (61.0 g) |
| divinyl benzene | (1.2 g) |
| 3-fluorostyrene | (5.0 g) |
| styrene | (32.5 g) |
| Luperox 26 | (1.5 g) |

Luperox 26 is hexaneperoxoic acid, 2-ethyl-1,1-dimethyl ethyl ester (CAS 3006-824). Luperox 26 is a trade mark of Elf Atochem.

The temperature was raised to 75° C. and maintained for 4% hours then raised to 80° C. for ½ hour to complete the polymerisation. The mixture was then cooled to <30° C.

Polymer Recovery

The product was filtered through a No. 2 sintered funnel using a water vacuum pump and washed with distilled water (˜5 litres) to remove any inorganics and emulsion polymer formed. The product was washed with propan-2-ol (3×500 ml) stirring after each addition. It was then washed with THF (3×500 ml) stirring after each addition to swell the beads. The swollen beads were then washed with diethyl ether (3×500 ml). The product was transferred to a crystallising dish and shaken periodically to prevent the beads forming agglomerates whilst the ether evaporated. When dry the beads were placed in a vacuum oven at 60° C. for ˜30 min to remove any remaining ether

Examples 2-5 illustrate the incorporation of secondary labels into the intrinsically labelled solid supports. The incorporation of the secondary label is illustrated without reference to library synthesis, however according to the invention the secondary labels would be introduced during library synthesis.

### EXAMPLE 2

Transformation of Intrinsically-Labelled poly(4-bromostyrene) into Intrinsic/Secondary-Labelled poly(4-(4′-chlorophenyl)styrene).

A mixture of poly(4-bromostyrene) 1.1 meq/g (0.5 g) and DME (6 ml) was stirred under argon and saturated aqueous sodium carbonate solution (0.63 ml), solid 4-chlorophenylboronic acid (122 mg) and tetrakis(triphenylphosphine)palladium (0) (29 mg) added. The reaction mixture was heated to reflux and stored at that temperature overnight, the mixture was diluted with DME (4 ml) and 25% aqueous ammonia solution (5 ml). The resin was removed by filtration and washed thoroughly with, in turn, DME, DME (aq), 0.2N HCl, THF, THF/triethylamine, THF, THF (aq), methanol and ether, then dried under vacuum to give poly(4-(4′-chlorophenyl)styrene) with a 70% incorporation of chlorine (0.51 g).

Found: C, 88.4%; H, 7.2%; N, 0%; Cl, 2.6%; Br, 0% (chlorine content 0.7 mEq/g).

### EXAMPLE 3

Transformation of Intrinsically-Labelled poly(4-bromostyrene) into Intrinsic/Secondary-Labelled poly(4-(4′-trifluoromethylphenyl)styrene).

A mixture of potassium fluoride (44 mg), water (0.6 ml), DME (4 ml), 4-trifluoromethylphenylboronic acid (48 mg) and poly(4-bromostyrene) 1.1 mEq/g (250 mg) was stirred under argon and tetrakis(triphenylphosphine)palladium (0) (20 mg) added. The reaction mixture was heated to reflux and maintained at that temperature for 17 hours. Aqueous ammonia solution (5%, 0.5 ml) was added and the mixture stirred at room temperature for 20 min before the resin was removed by filtration and washed thoroughly with, in turn, DME, DME/5% ammonia (aq), DME, DME/0.2N HCl, DME, methanol, THF, methanol and ether, then dried under vacuum to give poly(4-(4′-trifluoromethyl-phenyl)styrene) (263 mg) with a conversion of approximately 70%.

Found: C, 86.1%; H, 7.3%; N, 0%; Br, 2.7%.

### EXAMPLE 4

Transformation of Intrinsically-Labelled poly(4-bromostyrene) into Intrinsic/Secondary-Labelled poly(4-(4′-methoxyphenyl)styrene).

In a similar fashion to Example 2, poly(4-bromostyrene) (250 mg) and 4-methoxyphenylboronic acid (38 mg) gave poly(4-(4′-methoxyphenyl)styrene) (157 mg) with a conversion of approximately 97%.

Found: C, 89.3%; H, 7.7%; N. 0%; Br, 0.2%.

### EXAMPLE 5

Transformation of Intrinsically-Labelled poly(4-bromostyrene) into Intrinsic/Secondary-Labelled poly(4-(1′-naphthyl)styrene).

In a similar fashion to Example 2, poly(4-bromostyrene) (250 mg) and 1-naphthylboronic acid (43 mg) gave poly(4-(1′naphthyl)styrene) (267 mg) with a conversion of approximately 70%.

Found: C, 88.7%; H, 7.2%; N, 0%; Br, 2.0%.

### EXAMPLE 6

Identification of Intrinsic/Secondary Labels.

Single beads of the polybromostyrene and the corresponding para trifluoromethylphenyl labelled resin (as disclosed in Example 3) were heated separately in the source of a Vacuum Generators (Fisons) 70-250 mass spectrometer and a single spectrum was generated by summing several spectra collected over the period in time when the resin decomposed. The polybromostyrene resin showed clear peaks at m/z 182 and 184 corresponding to bromostyrene containing Br79/81 isotopes along with the confirmatory ion pair 13 mass units lower at m/z 169 and 171 corresponding to the bromotropylium ion pair. The trifluoromethylphenyl labelled resin showed the mono-isotopic molecular ion at m/z 248 for trifluoromethylphenylstyrene and the corresponding benzyl/tropylium ion at m/z 235.

A single resin bead containing chloromethylstyrene and fluorostyrene (as disclosed in Example 1) was also analysed. This showed the expected signature for fluorostyrene at m/z 122 and chloromethylstyrene at m/z 152(C135) and 154(C137) with the confirmatory ions at m/z 139 and 141.

1. A compound library comprising a plurality of different units each comprising a solid support with which is associated a single member of the compound library, characterised in that each solid support has a defined chemical composition which acts as an intrinsic label capable of identifying the first reaction choice in the synthesis of the associated member of the compound library.

2. A compound library according to claim 1, wherein the solid support comprises a polystyrene resin.

3. A compound library according to claim 2, wherein the solid support comprises chloromethylpolystyrene.

4. A compound library according to any one of the preceding claims, wherein the intrinsic label comprises at least one substituted styrene incorporated into the chemical composition of the solid support.

5. A compound library according to claim 4, wherein the substituted styrene is selected from halostyrenes and protected hydroxystyrenes.

6. A compound library according to any one of the preceding claims, wherein the solid support contains up to 20% w/w of the intrinsic label.

7. A compound library according to any one of the preceding claims, wherein the solid supports have one or more inert secondary labels which are introduced during library synthesis by controlled chemical modification of the solid support.

8. A method for the synthesis of a compound library comprising a plurality of different units each comprising a solid support with which is associated a single member of the compound library, which method comprises:
a) apportioning solid supports among a plurality of reaction vessels such that each reaction vessel contains a portion of solid support having a unique defined chemical composition which acts as an intrinsic label capable of identifying the first reaction choice in the synthesis of the associated member of the compound library;
b) exposing the supports in each reaction vessel to a first reaction choice;
c) pooling the supports;
d) apportioning the supports among a plurality of reaction vessels;
e) exposing the supports in each reaction vessel to a further reaction choice; and
f) repeating steps c), d) and e) as required.

9. A method according to claim 8, further comprising the introduction, during library synthesis, of at least one secondary inert label by controlled chemical modification of the solid support.

10. A method according to claim 9, wherein the secondary inert label is introduced by chemical modification of the intrinsic label.

11. A method for the characterisation of members of a compound library comprising a plurality of different units each comprising a solid support with which is associated a single member of the compound library, which method comprises:
i) synthesising the library by a method comprising:
a) apportioning solid supports among a plurality of reaction vessels such that each reaction vessel contains a portion of solid support having a unique defined chemical composition which acts as an intrinsic label capable of identifying the first reaction choice in the synthesis of the associated member of the compound library;
b) exposing the supports in each reaction vessel to a first reaction choice;
c) pooling the supports;
d) apportioning the supports among a plurality of reaction vessels;
e) exposing the supports in each reaction vessel to a further reaction choice; and
f) repeating steps c), d) and e) as required;
ii) testing members of the compound library for a characteristic of interest;
iii) selecting library compounds of interest; and
iv) identifying the intrinsic labels of the associated solid supports and identifying the first reaction choice in the synthesis of such compounds by reference to said intrinsic labels.

12. A method according to claim 11, further comprising the introduction, during library synthesis, of at least one secondary inert label by controlled chemical modification of the solid support, identifying the secondary inert labels of the solid support associated with a library compound of interest and identifying an intermediate reaction choice in the synthesis of such compounds by reference to said secondary inert labels.

13. A method according to claim 11 or 12, wherein the labels are excised from the resin by depolymerisation prior to identification.

14. A method according to claim 13, wherein the labels are excised from the resin by thermal depolymerisation.

15. A method according to any one of claims 11 to 14, wherein the labels are identified by gas chromatography, mass spectrometry or a combination of gas chromatography and mass spectrometry.

16. A method according to any one of claims 11 to 15, wherein the solid supports are divided into sections prior to identification of the labels.

17. A method according to claim 16, wherein the solid supports are divided into sections by microtomy.

18. A kit of solid supports for compound library synthesis comprising a plurality of portions of a solid support upon which associated members of a compound library can be synthesised, characterised in that each portion of the solid support has a unique defined chemical composition which acts as an intrinsic label capable of identifying the first reaction choice in the synthesis of the associated member of the compound library.
